(19) United States
(12) Patent Application Publication
(10) Pub. No.: US 2011/0046227 A1
(43) Pub. Date: Feb. 24, 2011

(54) METHODS FOR CONCOMITANT ADMINISTRATION OF COLCHICINE AND A SECOND ACTIVE AGENT

(75) Inventor: Matthew Davis, Erwinna, PA (US)

Correspondence Address: CANTOR COLBURN LLP, 20 Church Street, 22nd Floor, Hartford, CT, 06103, US

(73) Assignee: MUTUAL PHARMACEUTICAL COMPANY, INC., Philadelphia, PA (US)

(21) Appl. No.: 12/545,120

(22) Filed: Aug. 21, 2009

(51) Int. Cl. A61K 31/165

(52) U.S. Cl. 514/629

(57) ABSTRACT

Methods for concomitant administration of colchicine together with one or more second active agents, e.g., atorvastatin, are disclosed. Such methods reduce the dangers commonly associated with such concomitant administration and provide additional benefits. Methods of notifying health care practitioners and patients regarding appropriate dosing for concomitant administration of colchicine together with second active agents are also provided.

### FIELD OF THE DISCLOSURE

This disclosure relates to methods allowing for the co-administration of colchicine together with one or more second active agents for therapeutic purposes with improved safety compared to prior methods of administration.

### BACKGROUND

Colchicine, chemical name (−)-N-[(7S,12aS)-1,2,3,10-tetramethoxy-9-oxo-5,6,7,9-tetrahydrobenzo[a]heptalen-7-yl]-acetamide, is an alkaloid found in extracts of Colchicum autumnale, Gloriosa superba, and other plants. It is a microtubule-disrupting agent used in the treatment of gout and other conditions that may be treated, relieved or prevented with anti-inflammatory treatment. Colchicine impairs the motility of granulocytes and can prevent the inflammatory phenomena that initiate an attack (or flare) of gout. Colchicine also inhibits mitosis, resulting in effects in cells with high turnover rates such as those in the gastrointestinal tract and bone marrow. The primary adverse side effects of colchicine therapy include gastrointestinal upset such as diarrhea and nausea.

Colchicine has a narrow therapeutic index. The margin between an effective dose and a toxic dose of colchicine is much narrower than that of many other widely used drugs. Consequently, actions that result in increased colchicine levels in patients receiving colchicine therapy are particularly dangerous. Co-administration of colchicine to patients along with certain other drugs can have the effect of increasing colchicine levels. Such drug-drug interactions with colchicine have been reported to result in serious morbid complications and, in some cases, death.

Colchicine is rapidly absorbed from the gastrointestinal tract. Peak concentrations occur in 0.5 to 2 hours. The drug and its metabolites are distributed in leukocytes, kidneys, liver, spleen and the intestinal tract. Colchicine is metabolized in the liver and excreted primarily in the feces with 10 to 20% eliminated unchanged in the urine.

Gout (or gouty arthritis) is a disease caused by a build up of uric acid in the joints. Such a build up is typically due to an overproduction of uric acid, or to a reduced ability of the kidney to excrete uric acid. Gout is characterized by excruciating, sudden, unexpected, burning pain, as well as by swelling, redness, warmness, and stiffness in the affected joint. Low-grade fever may also be present. A gout flare is a sudden attack of pain in affected joints, especially in the lower extremities, and most commonly in the big toe. In afflicted individuals, the frequency of gout flares typically increases over time. In this manner, gout progresses from acute gout to chronic gout, which involves repeated episodes of joint pain.

Colchicine can reduce pain in attacks of acute gout flares and also can be used beneficially for treating adults for prophylaxis of gout flares. Although its exact mode of action in the relief of gout is not completely understood, colchicine is known to decrease the inflammatory response to urate crystal deposition by inhibiting migration of leukocytes, to interfere with urate deposition by decreasing lactic acid production by leukocytes, to interfere with kinin formation and to diminish phagocytosis and subsequent inflammatory responses. Colchicine is also used in treatment of familial Mediterranean fever (FMF), the most common of the autoinflammatory syndromes and which is characterized by recurrent inflammatory attacks of fever and serositis. FMF is an inherited disorder usually occurring in people of Mediterranean origin, including Sephardic Jews, Arabs, Armenians and Turks. The first episode usually occurs in childhood or the teenage years. Typically, attacks last 12 to 72 hours and can vary in severity. The length of time between attacks is also variable. Without treatment to help prevent attacks and complications, a buildup of amyloid depositions (amyloidosis) in the body's organs and tissues may occur, which can lead to organ failure, e.g., kidney failure.

Daily colchicine is the mainstay of therapy for FMF, resulting in complete remission or marked reduction in the frequency, duration, and severity of attacks in most patients. In an appropriate dose it prevents amyloidosis, even if it fails to improve attacks. Colchicine is also effective in arresting and reversing renal amyloidosis. The adult dosage of colchicine is typically 1-2 mg a day, depending on the patient's response, but daily dosages of up to 3 mg are occasionally given to patients. In children, the dose of colchicine is adjusted according to their body weight or body surface area. The minimal dose is about 0.25 mg daily in children 1-2 years old. By age 6-7, children can be treated with a dose of 1.0 mg daily.

Cytochrome p450 (CYP) enzymes are agents of drug metabolism that are found in the liver, the gastrointestinal tract and other locations in the body. CYP enzymes occur in a variety of closely related proteins referred to as isozymes and different CYP isozymes may preferentially metabolize different drugs. The 3A family of CYP isozymes, particularly CYP3A4, is also known to be involved in many clinically significant drug-drug interactions, including those involving colchicine and second active agents. While drugs are often targets of CYP-mediated metabolism, some may also alter the expression and activity of such enzymes, thus impacting the metabolism of other drugs. The biotransformation of colchicine in human liver microsomes involves formation of 3-demethylcolchicine and 2-demethylcolchicine. As shown by experiments using antibodies against CYP3A4 and experiments using chemical inhibition of CYP3A4, this transformation is correlated with (and thus apparently mediated by) CYP3A4 activity.

P-glycoprotein (P-gp) is an ATP-dependent cell surface transporter molecule that acts as an ATPase efflux pump for multiple cytotoxic agents, including colchicine. P-gp actively pumps certain compounds, including drugs such as colchicine, out of cells. P-gp is encoded by the Adenosine triphosphate-binding cassette subfamily B member 1 (ABCB1) gene, also referred to as the multiple drug resistance 1 gene (MDR1).

Since colchicine acts intracellularly, the combined effects of CYP3A4 inhibition and P-gp inhibition by second active agents that also interact with CYP3A4 and P-gp can cause colchicine toxicity in patients taking what would be a safe dose of colchicine in the absence of concomitant second agent administration. Various studies of adverse reactions from exposure to multiple drugs have found that 6.5-23% of the adverse reactions result from drug-drug interactions. Unfortunately, each year a number of deaths occur as the direct result of patients adding a concomitant prescription pharmaceutical product to their existing medication regimen.

There accordingly remains a need for improved methods for administering colchicine to individuals who are concomitantly being treated with second active agents so as to reduce the possibility of colchicine toxicity while maintaining the sometimes life-saving advantages of being able to administer the two (or more) agents concomitantly. The present disclosure addresses this need and provides further advantages.

### SUMMARY

In one embodiment, a method of treating a patient with colchicine, comprises

administering an adjusted daily dosage amount of colchicine to the patient who is receiving concomitant administration of atorvastatin, wherein the atorvastatin is administered to treat an atorvastatin-responsive condition,

wherein the adjusted daily dosage amount of colchicine is 50% to 75% of an intended daily dosage amount of colchicine, and

wherein the intended daily dosage amount of colchicine is a daily dosage amount suitable for the patient if the patient were not receiving concomitant atorvastatin, wherein concomitant administration of atorvastatin is administration within 1 to 2 days of orally administering the second colchicine dosage amount.

### BRIEF DESCRIPTION OF THE DRAWINGS

FIG. 1 shows mean colchicine plasma concentrations following administration of single and multiple oral doses of colchicine 0.6 mg in healthy adults, N=13, Y axis=colchicine concentration, ng/mL, X axis=time in hours, ♦=day 1, ▪=day 25. See Example 1.

FIG. 2 shows a pharmacokinetic profile comparison of single-dose colchicine (0.6 mg, alone) and single-dose colchicine (0.6 mg) co-administered with steady-state atorvastatin in healthy adults, Y axis=colchicine concentration, ng/mL, X axis=time in hours, N=23, ♦=colchicine alone, □=colchicine plus atorvastatin. See Example 2.

These and other embodiments, advantages and features of the present invention become clear when detailed description is provided in subsequent sections.

### DETAILED DESCRIPTION

Disclosed herein are methods for safely administering colchicine concomitantly with a second active agent, wherein the second active agent is a CYP3A4 inhibitor, a P-gp inhibitor, or both. An exemplary second active agent is atorvastatin. It has now been discovered that certain reduced or limited colchicine dosage amounts, when administered with concomitantly administered recommended dosage amounts of atorvastatin, achieve plasma colchicine levels that are therapeutically effective, but are not significantly higher, and therefore not significantly more toxic, than plasma levels achieved by administration of manufacturers' recommended colchicine dosages in the absence of concomitant administration with atorvastatin. Thus, colchicine and atorvastatin can be administered concomitantly with improved safety when colchicine is administered as disclosed herein.

Without being held to theory, it has been hypothesized by the inventors herein that P-gp inhibition is more important in the elimination of colchicine than CYP3A4 inhibition. Based on their level of P-gp inhibition by atorvastatin, it was predicted that atorvastatin will increase colchicine concentrations when the drugs are coadministered.

Atorvastatin, Lipitor®, is indicated for the prevention of heart disease and hypercholesterolemia (Fredrickson Types IIa and IIb, Fredrickson Type IV, Fredrickson Type III, homozygous familial hypercholesterolemia, and heterozygous familial hypercholesterolemia). The main metabolic pathway for atorvastatin is the CYP3A4 enzyme.

In one embodiment, colchicine is administered to an individual suffering from a condition treatable with colchicine, and the concomitant atorvastatin is administered concurrently while the colchicine administration is reduced, or the individual has recently completed a dosing regimen of atorvastatin, in which case the colchicine administration may still be reduced for a period of time.

In one embodiment, disclosed herein is a method of administering colchicine and atorvastatin, wherein an individual is administered the colchicine according to a colchicine dosing regimen of a single starting colchicine dose of no more than about 0.6 mg colchicine, followed by either no additional colchicine doses within about 12, 24, 48, or 72 hours, or followed by at least one additional colchicine dose within 12 hours and no more frequently than once every hour (e.g., every 3, 4, 6, 8, or 12 hours). In this embodiment, each additional colchicine dose is specifically no greater than about 0.3 mg and the individual is an adult individual or a pediatric individual. Specifically, the starting colchicine dose is about 0.6 mg or about 0.3 mg, and each additional colchicine dose is about 0.3 mg. In one embodiment, when additional doses are administered, only two, three, or four additional colchicine doses are administered within about 24 hours. Specifically, the individual is an adult individual and the starting colchicine dose is about 0.6 mg, and each additional colchicine dose, if any, is about 0.3 mg. In one embodiment, only three additional colchicine doses are administered within about 24 hours.

In one embodiment, disclosed herein is a method of administering colchicine and atorvastatin, wherein an individual is administered the colchicine according to a colchicine dosing regimen of a single starting colchicine dose of no more than about 1.2 mg colchicine, followed by either no additional colchicine doses within about 12, 24, 48, or 72 hours, or followed by at least one additional colchicine dose within 12 hours and no more frequently than once every hour (e.g., every 3, 4, 6, 8, or 12 hours). In this embodiment, each additional colchicine dose is specifically no greater than about 0.3 mg or 0.6 mg and the individual is an adult individual or a pediatric individual. Specifically, the starting colchicine dose is about 0.6 mg or 1.2 mg, and each additional colchicine dose is about 0.3 mg or 0.6 mg. In one embodiment, when additional doses are administered, only two, three, or four additional colchicine doses are administered within about 24 hours. Specifically, the individual is an adult individual and the starting colchicine dose is about 1.2 mg, and each additional colchicine dose, if any, is about 0.3 mg or 0.6 mg. In one embodiment, only three additional colchicine doses are administered within about 24 hours.

A method of treating a patient with colchicine comprises administering an adjusted daily dosage amount of colchicine to the patient who is receiving concomitant administration of atorvastatin, wherein the adjusted daily dosage amount of colchicine is 50% to 75% of an intended daily dosage amount of colchicine, and wherein the intended daily dosage amount of colchicine is a daily dosage amount suitable for the patient if the patient were not receiving concomitant atorvastatin. Treating with colchicine is, for example, to prevent gout flares, to treat acute gout, or to treat familial Mediterranean fever. When the colchicine is administered to prevent gout flares, the adjusted daily dosage amount of colchicine may be reduced from a 0.6 mg twice daily intended dose to a 0.6 mg once daily adjusted dose or from a 0.6 mg once daily intended dose to a 0.6 mg adjusted dose every other day. In one embodiment, when treating is for acute gout, the intended daily dosage amount is 1.8, and the maximum adjusted daily dosage amount is 1.2 mg. In another embodiment, treating is for acute gout, the intended daily dosage amount is 2.4 to 4.8 mg and the maximum adjusted daily dosage amount is about two-third of the intended daily dosage amount. In yet another embodiment, treating is for familial Mediterranean fever and the daily dosage amount 1.2 to 2.4 mg for adults, 0.9 to 18. mg for children between 6 and 12 years of age, and 0.3 to 1.8 mg for children between 4 and 6 years of age, and the adjusted daily dose is 50 to 75% of the intended daily dose. In one embodiment, the atorvastatin is administered to the patient before the colchicine is administered to the patient, and wherein the administration of atorvastatin is terminated no more than about fourteen days prior to the initiation of colchicine administration. The method optionally further comprises carefully monitoring the individual for potential toxicity. Any 0.3 mg dose contemplated in this method can be a single 0.3 mg dosage form or one-half a 0.6 mg dosage form, e.g. one-half a 0.6 mg colchicine tablet or a 0.6 mg scored colchicine tablet.

Disclosed herein are specific dosage reductions of colchicine that improve safety when colchicine is co-administered with certain active agents. The dose of colchicine recommended for administration without co-administration of certain other active agents, such as CYP3A4 or P-gp inhibitors, is referred to as an intended daily dosage amount. The reduced or modified daily dosage amount determined from the experiments presented herein is referred to as an adjusted daily dosage amount. An adjusted daily dosage amount is thus a daily dosage amount that can be safely co-administered with a second active agent as disclosed herein. A dose adjustment is thus a dose of colchicine and does not include cessation of colchicine, that is, a dose of 0 mg of colchicine.

In these and other embodiments, the colchicine-responsive condition is gout (e.g., a gout flare in an acute gout sufferer), familial Mediterranean fever (FMF), thrombocytopenic purpura, pericarditis, scleroderma, or Behçet's disease. In some embodiments, the treatment with colchicine is either palliative or prophylactic. The gout may be acute gout, e.g. a gout flare, or chronic gout.

### Acute Gout

Acute gout, or gout flares, can be treated according to the following treatment schedule. This table indicates the original, or intended, dose, i.e., the dose of colchicine recommended absent concomitant administration of the drugs listed below. This table also presents the dose adjustment, or the recommended colchicine dose to be administered when the strong and moderate CYP3A4 and P-gp inhibitors are administered concomitantly with colchicine when the patient is being treated for acute gout, or an acute gout flare.

| | Colchicine Dose Recommendation | |
|---|---|---|
| | Original Intended | |
| Drug | Dose (Total Dose) | Dose Adjustment |
| Moderate CYP3A4 | Regimen Reduced by One Third | |
| Inhibitors | | |
| Atorvastatin | 1.2 mg (2 tablets) at the first | 1.2 mg (2 tablets) × 1 dose. |
| | sign of the flare followed by 0.6 | Dose to be repeated no |
| | mg (1 tablet) one hour later. | earlier than 3 days. |
| | Dose to be repeated no earlier | |
| | than 3 days. | |

### Chronic Gout

For chronic gout, an original intended daily dosage amount is 1.2 mg or 6 mg. Alternatively, an intended daily dosage amount of chronic gout can be as much as 2.4 mg per day. The daily dosage amount for chronic gout can be administered at one time or dosed at intervals throughout the day, e.g. twice daily, three times daily, or four times daily.

Chronic gout, with and without a concomitant dose of another drug, can be treated according to the following treatment schedule:

### Colchicine Dose Adjustment for Co-Administration with Interacting Drugs if No Alternative Available

| | Colchicine Dose Recommendation | |
|---|---|---|
| Drug | Original Intended Dose | Dose Adjustment |
| Atorvastatin | 0.6 mg twice daily | 0.6 mg once daily |
| | 0.6 mg once daily | 0.6 mg once every other day |

### Familial Mediterranean Fever

Familial Mediterranean Fever (FMF) can be treated according to the following intended daily dosing schedule:

Daily dosage amount

| Age | Usual | Maximum |
|---|---|---|
| Adults and children >12 years | 1.2 mg | 2.4 mg |
| Children >6 to 12 years | 0.9 mg | 1.8 mg |
| Children 4 to 6 years | 0.3 mg | 1.8 mg |

When colchicine is given to patients with FMF concomitantly with other drugs, the adjusted (reduced) dosage amount of colchicine, according to this embodiment, is provided in the table below:

| Concomitant Drug | Noted or Anticipated | |
|---|---|---|
| Class or Food | Outcome | Clinical Comment |
| Moderate CYP3A4 | Significant increase in | Use colchicine with |
| inhibitors: | colchicine plasma | caution at reduced |
| Atorvastatin | concentration is | maximum dose of 0.6 mg |
| | anticipated. | twice daily with increased |
| | | monitoring for adverse |
| | | effects. In patients with |
| | | renal or hepatic |
| | | impairment, use a |
| | | maximum dose of 0.3 mg |
| | | twice daily. |

Pharmacy management systems are computer-based systems that are widely used by commercial pharmacies to manage prescriptions and to provide pharmacy and medical personnel with warnings and guidance regarding drugs being administered to individuals. Such systems typically provide alerts warning either or both of health care providers and patients when a drug that may be harmful to the particular patient is prescribed. For example, such systems can provide alerts warning that a patient has an allergy to a prescribed drug, or is receiving concomitant administration of a drug that can have a dangerous interaction with a prescribed drug. U.S. Pat. Nos. 5,758,095, 5,833,599, 5,845,255, 6,014,631, 6,067,524, 6,112,182, 6,317,719, 6,356,873, and 7,072,840, each of which is incorporated herein by reference, disclose various pharmacy management systems and aspects thereof. Many pharmacy management systems are now commercially available, e.g., CENTRICITY Pharmacy from BDM Information Systems Ltd., General Electric Healthcare, Waukesha, Wis., Rx30 Pharmacy Systems from Transaction Data Systems, Inc., Ocoee, Fla., SPEED SCRIPT from Digital Simplistics, Inc., Lenexa, Kans., and various pharmacy management systems from OPUS-ISM, Hauppauge, N.Y.

In another aspect, herein disclosed are methods for using colchicine which methods involve the use of pharmacy management systems.

In one aspect, one such method comprises a pharmacy receiving a prescription for colchicine for a patient who is suffering from gout (e.g., acute gout flares or chronic gout) and who is concomitantly being treated with a second active agent (e.g., atorvastatin) that is an inhibitor of CYP3A and P-glycoprotein, followed by the pharmacy dispensing colchicine in response to receipt of the prescription, wherein the dispensing is preceded by entry into a first computer readable storage medium, in functional communication with a computer, of a unique patient identifier for said patient and at least one drug identifier for colchicine linked to the patient identifier so as to indicate that colchicine is to be administered to the patient. The computer is programmed to issue a drug-drug interaction alert when the at least one drug identifier for colchicine is entered linked to the patient identifier so as to indicate that colchicine is to be administered to the patient and when the patient identifier is also linked to an identifier indicating that a second active agent (e.g., atorvastatin) that is an inhibitor of CYP3A4 or P-glycoprotein is being concomitantly administered to the patient. Upon entry of the at least one drug identifier for colchicine linked to the patient identifier, a drug-drug interaction alert is issued to one or more of a pharmacy technician, a pharmacist, or a pharmacy customer obtaining the colchicine, said alert indicating that a second active agent (e.g., atorvastatin) is being concomitantly administered to the patient and that prior to the colchicine being dispensed, the colchicine dosing regimen must be reviewed and, if necessary adjusted, so that when the colchicine is delivered to the pharmacy customer obtaining the colchicine it is delivered along with instructions for the colchicine to be taken in accordance with a dosing regimen of no more than one about 1.2 mg colchicine dose to start (e.g., following the onset of the acute gout attack or the first sign of a gout flare) followed by either: no additional colchicine doses within about 12, 24, 48, or 72 hours, or at least one additional colchicine dose within about 12 hours and no more frequently than once every hour and wherein each additional colchicine dose is no greater than about 0.6 mg, and wherein the patient ingests the colchicine as instructed.

The drug-drug interaction alert is preferably issued as one or both of a written warning on a display screen of the pharmacy management computer system, and a printed warning. The printed warning may be attached to or packaged with the dispensed prescription.

In one aspect, the identifier indicating that atorvastatin is being concomitantly administered to the patient is an identifier indicating that the second active agent is atorvastatin and is linked to at least one further identifier indicating that the atorvastatin is prescribed so that 10, 20, 40 or 80 mg of atorvastatin is to be ingested by the patient daily, in which case the dosing regimen for colchicine is preferably one about 0.6 mg or 1.2 mg colchicine dose to start for acute gout patients or chronic gout patients, optionally followed by additional colchicine doses, e.g., 0, 1, 2, 3, or 4 additional colchicine doses within 24 hours of about 0.3 mg or 0.6 mg ingested every 1, 2, 3, 4, 5, 6, 7, 8, 9 10, 11, or 12 hours (e.g., every 2, 3, 4, 5, or 6 hours) after the preceding colchicine dose. In another embodiment, the identifier indicating that atorvastatin is being concomitantly administered to the patient is an identifier indicating that the second active agent is atorvastatin is linked to at least one further identifier, entered into a second computer readable storage medium in functional communication with a computer, the second storage medium being the same as or different from the first storage medium, and the further identifier indicating that the atorvastatin is prescribed so that about 10, 20, 40 or 80 mg of atorvastatin is to be ingested by the patient daily, in which case the colchicine dosing regimen is one about 0.6 mg or 1.2 mg colchicine dose to start, followed by an about 0.3 mg or 0.6 mg colchicine dose ingested every 1, 2, 3, 4, 5, 6, 7, 8, 9, 10, 11, or 12 hours (e.g., every six to eight hours) after the preceding colchicine dose. In one embodiment, the dosing regimen calls for the about 0.3 mg colchicine dose every six to eight hours. In another embodiment, the dosing regimen calls for one dose of the colchicine every eight to twelve hours.

In yet another preferred aspect, the identifier indicating that atorvastatin is being concomitantly administered to the patient is an identifier indicating that the second active agent is atorvastatin and is linked to at least one further identifier, entered into a second computer readable storage medium in functional communication with a computer, the second storage medium being the same as or different from the first storage medium, and the further identifier indicating that the atorvastatin is prescribed so that about 10, 20, 40 or 80 mg of atorvastatin is to be ingested by the patient daily and the dosing regimen is one about 0.3 mg colchicine dose to start, followed by an about 0.3 mg colchicine dose ingested every 2, 3, 4, 5, 6, 7, or 8 hours (e.g., every eight to twelve hours) after the preceding colchicine dose.

A preferred dosing regimen calls for ingestion of colchicine to be continued until a total of no more than 1.2 mg or 2.4 mg of colchicine has been ingested, after which ingestion of colchicine is to be stopped, e.g., for at least 2, 3, 4, 5, 6, or 7 days, or until a subsequent acute gout flare, or the first sign of a subsequent gout flare, occurs.

Also disclosed herein is a dosage adjustment method for administering colchicine to a patient to treat a medical condition, the patient concomitantly treated with a second active agent. The second active agent is, for example, atorvastatin. The method comprises determining a first colchicine dosing regimen (the colchicine dosing regimen suitable for administration in the absence of co-administration with a second active agent, which dosing regimen may consist of one or more doses of colchicine); and determining a second active agent dosing regimen; and administering the second active agent to the patient at the second active agent dosing regimen while concomitantly administering colchicine to the patient according to a second colchicine dosing regimen, which may consist of one or more reduced colchicine doses. The second colchicine dosing regimen is a fraction of the first colchicine dosing regimen, where the fraction is obtained by administering reduced colchicine doses or by reducing the frequency of colchicine doses, and wherein the fraction is less than or equal to about ¾ or less than or equal to about ⅔ or less than or equal to about ½.

According to this embodiment, upon the administering the second active agent to the patient at the second active agent dosing regimen while concomitantly administering colchicine to the patient at the second colchicine dosing regimen, the therapeutic circulating level of colchicine is achieved in the patient. Preferably, the fraction is selected from ½, ⅔, ¾, (any others?), more preferably, the fraction is ⅔ or ¾. In one embodiment, if the second colchicine dosing regimen comprises a “first” colchicine dose, and one or more “subsequent” colchicine doses, each subsequent colchicine dose may be the same as the first dose, or a fraction of the first dose. The fraction is selected from about ½, about ⅔, about ¾. In one example, the second colchicine dosing regimen is once-a-day, twice-a-day, three-times-a-day or four-times-a-day. In a variation of this example, the initial treatment day in, a second colchicine dosing regimen that lasts for more than one day, has one more dose administered than are administered each subsequent day.

The specific conditions are selected from gout, FMF, thrombocytopenic purpura, and Behçet's disease. In one embodiment, the gout is an acute gout flare and the colchicine treatment schedule is an acute treatment schedule adapted for treatment of acute gout flares, or the gout is chronic gout, and the colchicine treatment schedule is a chronic treatment schedule adapted for prophylaxis, or prevention, of flares. In another embodiment, the fraction of colchicine administered to the patient concomitantly with a second active agent that is a CYP3A4 or P-gp inhibitor is ½ or 34 the original intended amount of colchicine and treatment with colchicine is initiated subsequent to or at the same time as initiation of treatment with the second active agent.

Colchicine is one of the most widely used drugs for treating familial Mediterranean fever (FMF). It has been reported that 5-10% of FMF patients do not show a beneficial response to colchicine administration. A polymorphism in the ABCB1 gene, the “ABCB1 3435 C to T polymorphism” has been reported to correlate with this lack of response to colchicine treatment, with patients with the homozygous TT genotype exhibiting the most pronounced “non-responder” phenotypes.

Accordingly, in another aspect, provided herein is a method for treating a patient suffering from FMF, which patient is a colchicine non-responder. Preferably, the patient is homozygous for the TT genotype of the ABCB1 3435 C to T polymorphism. The method entails the concomitant administration of a P-gp inhibitor and colchicine to the patient. Exemplary P-gp inhibitors include atorvastatin. Preferred dosages of the P-gp inhibitor for this purpose correspond to those called for in the prescribing information for the drug in question. For atorvastatin, an exemplary dosage is 10, 20, 40 or 80 mg. Specific colchicine dosing regimens for this purpose are the same as used for treatment of FMF in responders, though the doses of colchicine administered may be increased as tolerated, e.g., up to two to three times the typical doses.

The following examples further illustrate aspects of this disclosure but should not be construed as in any way limiting its scope. In particular, the conditions are merely exemplary and can be readily varied by one of ordinary skill in the art.

### EXAMPLES

### Example 1

### Pharmacokinetic Study in Healthy Adults of Single vs. Multiple Oral Doses of Colchicine Tablets

This study was a single-center, open-label, single-sequence, two-period study to evaluate the pharmacokinetic profile of colchicine following single and multiple oral doses of colchicine tablets, 0.6 mg, in healthy volunteers.

In Period 1, study subjects received a 0.6-mg dose of colchicine after an overnight fast of at least 10 hours. In Period 2, subjects received a 0.6 mg dose of colchicine in the morning and the evening (approximately 12 hours later) for 10 days (steady state regimen). Subjects received a light breakfast served 60 minutes following dose administration in the morning and the evening dose was administered 90 minutes after an evening meal on Days 15 through 24 only. On Day 25, the colchicine dose was administered after an overnight fast of at least 10 hours and lunch was served 4 hours post-dose. Study periods were separated by a 14-day washout. Following the single dose and the last dose of the multiple dose regimen (beginning on the mornings of Day 1 and Day 25, respectively), blood samples were collected (6 mL each) from each subject within 1 hour prior to dosing and after dose administration at study hours 0.5, 1, 1.5, 2, 3, 4, 6, 8, 10, 12, and 24 (while confined) and 36, 48, 72, and 96 (on an outpatient basis). Plasma concentrations of colchicine and its metabolites were determined using validated LC/MS-MS methods.

Thirteen healthy, non-smoking subjects with a mean age of 25.5 years (range 19 to 38 years) and within 15% of ideal body weight were enrolled in this study. All subjects completed both dosing periods according to protocol.

After a single dose, plasma concentrations are no longer quantifiable 24 hours post-dose in all but 1 subject. After the last dose of the steady state regimen, concentrations remained quantifiable for 48 to 72 hours. Review of individual subject data shows that no subject experienced a secondary colchicine peak, either following a single dose or upon multiple dosing.

All 2-O-demethylcolchicine (2-DMC) concentrations were below the level of quantitation (LOQ, 0.2 ng/mL) and only one sample from 1 subject (of 13 subjects) had a detectable 3-O-demethylcolchicine (3-DMC) concentration, which was near the level of quantitation. Therefore, metabolites are not discussed further.

In healthy adults, colchicine appears to be readily absorbed when given orally, reaching a mean maximum plasma concentration of 2.5 ng/mL in 1.5 hours after a single dose. The drug is distributed widely, with an apparent volume of distribution of 540 L, greatly exceeding total body water. The elimination half-life as calculated following a single oral dose is approximately 5 hours. Levels were not detectable by 24 hours post-dose and this is therefore not an accurate estimate. Pharmacokinetic parameter values are summarized in the table below.

Review of trough plasma concentrations indicates that steady state was attained by approximately the eighth day of dosing for most subjects. Colchicine may have a diurnal variation reflected in the observed Cmin concentrations at steady state. Cmin concentrations prior to the morning dose are approximately 12% higher than the Cmin concentrations prior to the evening dose (Day 23 and Day 24). The mean Cmin concentration observed on Day 25 was 0.907 ng/mL.

Colchicine accumulated following administration of multiple doses to an extent greater than expected. Exposure was nearly two-fold higher (approximately 1.7 based on AUC [Day 25 AUC_{0-τ}/Day 1 AUC_{0-∞}] and approximately 1.5 based on Cmax [Day 25 Cₘₐₓ/Day 1 Cₘₐₓ]). This observation could be attributable to an underestimation of AUC∞ following a single dose. With the higher plasma levels that occur with repeated dosing, a longer terminal elimination half life is apparent, 26.6 hours. Pharmacokinetic parameter values are summarized in Tables 3-5.

TABLE 3
Colchicine Pharmacokinetic Parameter Values Following Administration
of A Single Oral Dose of Colchicine 0.6 mg in Healthy Adults (N = 13)

| | MEAN | STDEV | % CV | MEDIAN | MIN | MAX |
|---|---|---|---|---|---|---|
| AUC₀₋ₜ | 10494.66 | 3544.08 | 33.77 | 10560.90 | 4812.88 | 18091.85 |
| (pg-hr/mL) | | | | | | |
| AUC_{0-inf} | 12268.18 | 4422.08 | 36.05 | 11451.45 | 7252.66 | 23801.68 |
| (pg-hr/mL) | | | | | | |
| Cmax | 2450.15 | 702.11 | 28.66 | 2480.00 | 1584.00 | 3977.00 |
| (pg/mL) | | | | | | |
| Tmax | 1.50 | 0.54 | 36.00 | 1.50 | 1.00 | 3.00 |
| (hr) | | | | | | |
| Kₑₗ | 0.1829 | 0.0592 | 32.38 | 0.1992 | 0.0359 | 0.2443 |
| (1/hr) | | | | | | |
| T_{1/2} | 4.95 | 4.43 | 89.54 | 3.48 | 2.84 | 19.29 |
| (hr) | | | | | | |

TABLE 4
Colchicine Pharmacokinetic Parameter Values Following Administration of Multiple
(b.i.d.) Oral Doses of Colchicine 0.6 mg in Healthy Adults (N = 13)

| | MEAN | STDEV | % CV | MEDIAN | MIN | MAX |
|---|---|---|---|---|---|---|
| AUC₀₋ₜ | 43576.96 | 9333.26 | 21.42 | 41925.10 | 29328.78 | 58265.35 |
| (pg-hr/mL) | | | | | | |
| AUC_{0-τ} | 20366.61 | 3322.12 | 16.31 | 20423.08 | 13719.18 | 25495.25 |
| (pg-hr/mL) | | | | | | |
| AUC_{0-inf} | 54198.77 | 9214.54 | 17.00 | 54113.43 | 37599.76 | 67944.65 |
| (pg-hr/mL) | | | | | | |
| Cₘₐₓ | 3553.15 | 843.45 | 23.74 | 3734.00 | 1977.00 | 4957.00 |
| (pg/mL) | | | | | | |
| Cₘᵢₙ | 906.51 | 152.19 | 16.79 | 903.50 | 636.23 | 1149.67 |
| (pg/mL) | | | | | | |
| Cₐᵥₑ | 1697.22 | 276.84 | 16.31 | 1701.92 | 1143.26 | 2124.60 |
| (pg/mL) | | | | | | |
| Tₘₐₓ | 1.31 | 0.60 | 45.61 | 1.00 | 0.50 | 3.00 |
| (hr) | | | | | | |
| Kₑₗ | 0.0267 | 0.0044 | 16.34 | 0.0261 | 0.0206 | 0.0333 |
| (1/hr) | | | | | | |
| T_{1/2} | 26.60 | 4.33 | 16.26 | 26.51 | 20.82 | 33.65 |
| (hr) | | | | | | |

TABLE 5
Mean (% CV) Colchicine Pharmacokinetic Parameter Values
Following Administration of Single and Multiple (b.i.d.)
Oral Doses of Colchicine 0.6 mg in Healthy Adults

| | |
|---|---|
| Vd/F (L) | CL/F (L/hr) |

Colchicine 0.6-mg Single Dose (N = 13)

| | | |
|---|---|---|
| Day 1 | 341 (54.4) | 54.1 (31.0) |

Colchicine 0.6 mg b.i.d. × 10 days

| | | |
|---|---|---|
| Day 25 | 1150 (18.73) | 30.3 (19.0) |

CL = Dose/AUC₀₋ₜ (Calculated from mean values)
Vd = CL/Ke (Calculated from mean values)

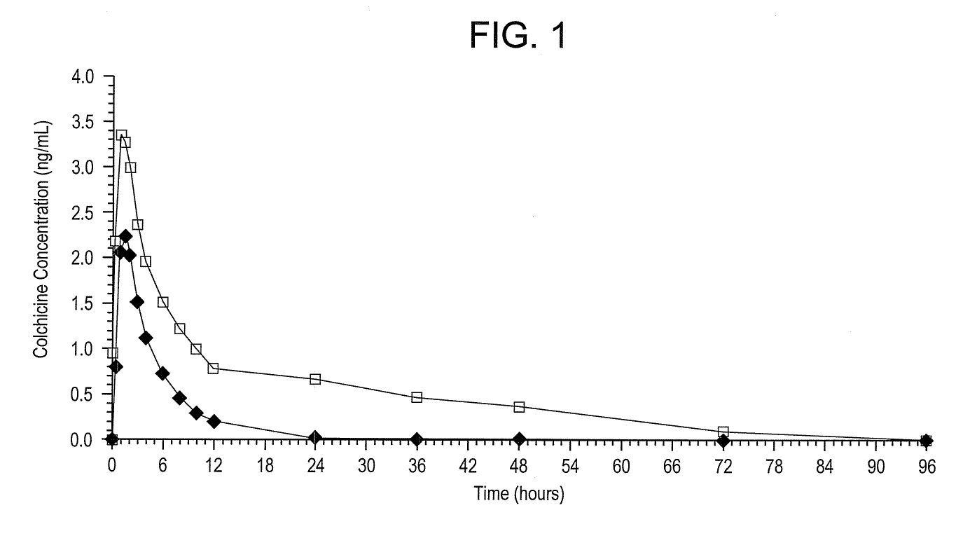

In tables, the parameter CL/F denotes the apparent total body clearance after administration, calculated as Total Dose/Total AUC0-ₜₐᵤ; and V_{d}/F denotes the apparent total volume of distribution after administration, calculated as Total Dose/(Total AUC_{∞}×Kₑₗ). FIG. 1 shows mean colchicine plasma concentrations following administration of single and multiple oral doses of colchicine 0.6 mg in healthy adults.

### Example 2

### Clinical Drug-Drug Interaction Study of Colchicine and Atorvastatin

This study was an open-label, non-randomized, single-center, one-sequence, two-period drug interaction study conducted in healthy male and female volunteers. Twenty-four (24) non-smoking, non-obese adult volunteers were enrolled. All subjects were dosed and studied as a single cohort, with each subject receiving the same treatment in a non-randomized fashion.

A single dose of colchicine, 0.6 mg, was administered alone on Day 1, and then co-administered with atorvastatin on Day 28. Atorvastatin was administered [40 mg once daily] beginning on the morning of Day 15, with the last 40-mg atorvastatin dose administered on the morning on Day 28. A 14-day washout period was completed after the first colchicine dose on Day 1 and prior to the administration of the first atorvastatin dose on Day 15.

Serial blood samples were collected by individual venipuncture up to 24 hours following drug(s) administration on Day 1 and Day 28. Blood samples for determination of colchicine plasma concentrations were obtained at time zero (pre-dose) and after dose administration at 0.5, 1.0, 1.5, 2, 3, 4, 5, 6, 8, 12, and 24 hours post-dose on an inpatient basis. Atorvastatin plasma concentrations were not measured.

TABLE 6
Comparison of Single-Dose Colchicine (0.6 mg, Alone)
and Single-Dose Colchicine (0.6 mg) Co-Administered
with Steady-State Atorvastatin in Healthy Adults

| | | Colchicine + | |
|---|---|---|---|
| | Colchicine Alone | Atorvastatin | % Ratio |
| Cₘₐₓ (pg/mL), geometric | 1842.2 | 2406.9 | 130.6 |
| mean | | | |
| AUC₀₋ₜ (pg · h/mL), | 7906.83 | 10074.5 | 127.41 |
| geometric mean | | | |
| AUC_{∞} (pg · h/mL), | 9063.0 | 11275.0 | 124.40 |
| geometric mean | | | |

TABLE 7
Comparison of Single-Dose Colchicine (0.6 mg, Alone)
and Single-Dose Colchicine (0.6 mg) Co-Administered
with Steady-State Atorvastatin in Healthy Adults

| | Arithmetic Mean (% CV) | |
|---|---|---|
| | Median (Range) for Tₘₐₓ | |
| | Colchicine + Atorvastatin | Colchicine Alone |
| Parameter (units) | (N = 23) | (N = 23) |
| AUC₀₋ₜ (ng · hr/mL) | 10716.3 | 8711.94 |
| AUC_{0-∞} (ng · hr/mL) | 11649.5 | 9571.6 |
| Cₘₐₓ (ng/mL) | 2487.9 | 2021.6 |
| Tₘₐₓ (hr) | 1.21 | 1.23 |
| t_{1/2} (hr) | 4.30 | 3.783 |
| CL/F (L/hr) | 54400.3 | 69990.91 |

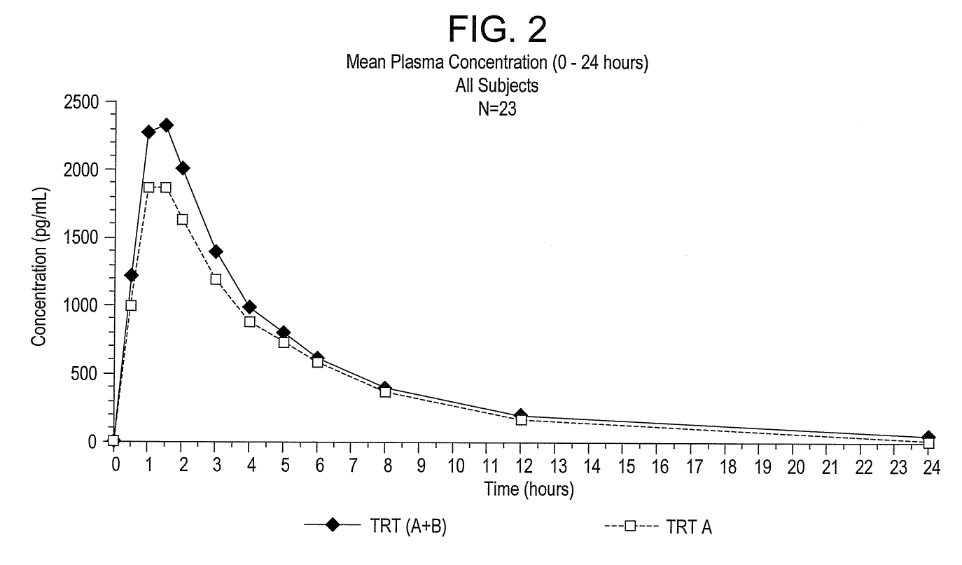

FIG. 2 shows a pharmacokinetic profile comparison of single-dose colchicine (0.6 mg, alone) and single-dose colchicine (0.6 mg) co-administered with steady-state atorvastatin in healthy adults. Based on the foregoing data, when coadministered with atorvastatin, the dose of colchicine should be reduced by ¼ to ½, specifically ⅓ to ½.

Recitation of ranges of values herein are merely intended to serve as a shorthand method of referring individually to each separate value falling within the range, unless otherwise indicated herein, and each separate value is incorporated into the specification as if it were individually recited herein. The endpoints of all ranges directed to the same component or property are inclusive and independently combinable.

All methods described herein can be performed in a suitable order unless otherwise indicated or clearly contradicted by context. The use of any and all examples, or exemplary language (e.g., “such as”) herein is intended to better illuminate the disclosure and is non-limiting unless otherwise specified. No language in the specification should be construed as indicating that any non-claimed element as essential to the practice of the claimed embodiments. Unless defined otherwise, technical and scientific terms used herein have the same meaning as is commonly understood by one of skill in the art to which this disclosure belongs. The terms wt %, weight percent, percent by weight, etc. are equivalent and interchangeable.

In the specification and claims that follow, references will be made to a number of terms which shall be defined to have the following meaning.

The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

“Concomitant” and “concomitantly” as used herein refer to the administration of at least two drugs to a patient either simultaneously or within a time period during which the effects of the first administered drug are still operative in the patient. Thus, if the first drug is, e.g., atorvastatin and the second drug is colchicine, the concomitant administration of the second drug can occur as much as one to two weeks, preferably within one to seven days, after the administration of the first drug. This is because atorvastatin can exert an inhibition of CYP3A isozymes so that CYP3A activity in the patient may not return to pre-atorvastatin-administration levels for as much as two weeks after the cessation of atorvastatin administration. If colchicine is the first drug, administration of a second drug would be concomitant if done within 1 to 2 days, preferably 12 to 24 hours.

A “dose” means the measured quantity of a drug to be taken at one time by a patient.

A “dosage amount” means an amount of a drug suitable to be taken during a fixed period, usually during one day (i.e. daily). A “daily dosage amount” is the total dosage amount taken in one day, that is, a 24 hour period.

“Dosing regimen” means the dose of a drug taken at a first time by a patient and the interval (time or symptomatic) and dosage amounts at which any subsequent doses of the drug are taken by the patient. Each dose may be of the same or different.

A “patient” means a human or non-human animal in need of medical treatment. Medical treatment can include treatment of an existing condition, such as a disease or disorder, prophylactic or preventative treatment, or diagnostic treatment. In preferred embodiments the patient is human.

“Providing” means giving, administering, selling, distributing, transferring (for profit or not), manufacturing, compounding, or dispensing.

“Risk” means the probability or chance of adverse reaction, injury, or other undesirable outcome arising from a medical treatment. An “acceptable risk” means a measure of the risk of harm, injury, or disease arising from a medical treatment that will be tolerated by an individual or group. Whether a risk is “acceptable” will depend upon the advantages that the individual or group perceives to be obtainable in return for taking the risk, whether they accept whatever scientific and other advice is offered about the magnitude of the risk, and numerous other factors, both political and social. An “acceptable risk” of an adverse reaction means that an individual or a group in society is willing to take or be subjected to the risk that the adverse reaction might occur since the adverse reaction is one whose probability of occurrence is small, or whose consequences are so slight, or the benefits (perceived or real) of the active agent are so great. An “unacceptable risk” of an adverse reaction means that an individual or a group in society is unwilling to take or be subjected to the risk that the adverse reaction might occur upon weighing the probability of occurrence of the adverse reaction, the consequences of the adverse reaction, and the benefits (perceived or real) of the active agent. “At risk” means in a state or condition marked by a high level of risk or susceptibility.

Pharmacokinetic parameters referred to herein describe the in vivo characteristics of drug (or a metabolite or a surrogate marker for the drug) over time. These include plasma concentration (C), as well as Cₘₐₓ, Cₙ, C₂₄, Tₘₐₓ, and AUC. “Cₘₐₓ” is the measured plasma concentration of the active agent at the point of maximum, or peak, concentration. “Cₘᵢₙ” is the measured plasma concentration of the active agent at the point of minimum concentration. “Cₙ” is the measured plasma concentration of the active agent at about n hours after administration. “C₂₄” is the measured plasma concentration of the active agent at about 24 hours after administration. The term “Tₘₐₓ” refers to the time from drug administration until Cₘₐₓ is reached. “AUC” is the area under the curve of a graph of the measured plasma concentration of an active agent vs. time, measured from one time point to another time point. For example AUC₀₋ₜ is the area under the curve of plasma concentration versus time from time 0 to time t, where time 0 is the time of initial administration of the drug. Time t can be the last time point with measurable plasma concentration for an individual formulation. The AUC_{0-∞}, AUC_{∞} or AUC_{0-inf} is the calculated area under the curve of plasma concentration versus time from time 0 to time infinity. In steady-state studies, AUC_{0-τ} is the area under the curve of plasma concentration over the dosing interval (i.e., from time 0 to time τ (tau), where tau is the length of the dosing interval. Other pharmacokinetic parameters are the parameter Kₑ or Kₑₗ the terminal elimination rate constant calculated from a semi-log plot of the plasma concentration versus time curve; t_{1/2} the terminal elimination half-life, calculated as 0.693/Kₑₗ. CL/F denotes the apparent total body clearance after administration, calculated as Total Dose/Total AUC_{∞}; and Vₐᵣₑₐ/F denotes the apparent total volume of distribution after administration, calculated as Total Dose/(Total AUC_{∞}×Kₑₗ).

“Side effect” means a secondary effect resulting from taking a drug. The secondary effect can be a negative (unfavorable) effect (i.e., an adverse side effect) or a positive (favorable) effect.

The most frequently reported adverse side effects to colchicine therapy are gastrointestinal, specifically abdominal pain with cramps, diarrhea, nausea, and vomiting. Less frequently or rarely reported adverse side effects associated with colchicine therapy include anorexia, agranulocytosis, allergic dermatitis, allergic reactions, alopecia, angioedema, aplastic anemia, bone marrow depression, myopathy, neuropathy, skin rash, thrombocytopenic disorder, and urticaria.

Whether a patient experiences an adverse side effect can be determined by obtaining information from the patient regarding onset of certain symptoms which may be indicative of the adverse side effect, results of diagnostic tests indicative of the adverse side effect, and the like.

Embodiments are described herein, including the best modes known to the inventors. Variations of such embodiments will become apparent to those of ordinary skill in the art upon reading the foregoing description. The skilled artisan is expected to employ such variations as appropriate, and the disclosed methods are expected to be practiced otherwise than as specifically described herein. Accordingly, all modifications and equivalents of the subject matter recited in the claims appended hereto are included to the extent permitted by applicable law. Moreover, any combination of the above-described elements in all possible variations thereof is encompassed unless otherwise indicated herein or otherwise clearly contradicted by context.

1. A method of treating a patient with colchicine, comprising
orally administering an adjusted daily dosage amount of colchicine to the patient who is receiving concomitant administration of a daily dose of atorvastatin, wherein the atorvastatin is administered to treat an atorvastatin-responsive condition,
wherein the adjusted daily dosage amount of colchicine is about 50% to 75% of an intended daily dosage amount of colchicine, and
wherein the intended daily dosage amount of colchicine is a daily dosage amount suitable for the patient if the patient were not receiving concomitant atorvastatin, wherein concomitant administration of atorvastatin is administration either simultaneously with colchicine or within a time period during which the effects of the colchicine are still operative in the patient.

2. The method of claim 1, wherein treating with colchicine is treating chronic gout.

3. The method of claim 1, wherein treating with colchicine is treating for acute gout.

4. The method of claim 1, wherein treating with colchicine is treating familial Mediterranean fever.

5. The method of claim 3, wherein, the intended daily dosage amount is 1.8 mg/day and the adjusted daily dosage amount is 1.2 mg/day.

6. The method of claim 5, further comprising not repeating colchicine administration for at least three days.

7. The method of claim 2, wherein, the intended daily dosage amount is 1.2 mg/day and the adjusted daily dosage amount is 0.6 mg/day.

8. The method of claim 2, wherein, the intended daily dosage amount is 0.6 mg/day and the adjusted daily dosage amount is 0.6 mg every other day.

9. The method of claim 1, further comprising carefully monitoring the individual for potential toxicity.

10. The method of claim 3, wherein the intended daily dosage amount is 1.8 to 2.4 mg and the maximum adjusted daily dosage amount is 1.8 mg.

11. The method of claim 4, wherein the intended daily dosage amount is 1.2 to 2.4 mg and the maximum adjusted daily dosage amount is 0.6 mg.

12. The method of claim 4, wherein treating is for familial Mediterranean fever, the intended daily dosage amount is 0.9 to 1.8 mg and the maximum adjusted daily dosage amount is 0.6 mg.

13. The method of claim 4, wherein the intended daily dosage amount is 0.3 to 1.8 mg and the maximum adjusted daily dosage amount is 0.6 mg.
